(19) United States
(12) Patent Application Publication
(10) Pub. No.: US 2008/0181851 A1
(43) Pub. Date: Jul. 31, 2008

(54) PHOTOACOUSTIC CONTRAST AGENTS FOR MOLECULAR IMAGING

(76) Inventor: Samira Guccione, Hillsborough, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/959,376

(22) Filed: Dec. 18, 2007

Related U.S. Application Data
(60) Provisional application No. 60/870,573, filed on Dec. 18, 2006.

(51) Int. Cl. A61K 49/00

(52) U.S. Cl. 424/9.5

(57) ABSTRACT

Compositions of photoacoustic tomography (PAT) contrast agents, and methods of achieving contrast enhancement and amplification of photo-induced acoustic signal for in vivo photoacoustic imaging of animals and human subjects are provided. Contrast agents of interest are organic-based agents, which may be targeted or non-targeted, e.g. protein-based and/or lipid-based molecules, in combination with an inorganic core, e.g. a metal or silicon core, herein referred to as a composite PAT contrast agent. Preferred agents absorb in the near IR spectrum, e.g. from around about 650 nm to around about 800 nm, for example from about 720 to about 790 nm. In some embodiments of the invention, the organic component is a lipid composition, which optionally comprises one or more targeting moieties. In some embodiments the inorganic core is gold or another noble metal, e.g. silver, platinum, etc.

Photoacoustic tomography (PAT) is a multi-modality imaging technique that utilizes non-ionizing energy to obtain both structural and functional information. Although photoacoustic imaging is closely related to ultrasound there are important differences inherent in the use of optical energy to generate ultrasound waves. Perhaps the most significant difference is the fact that acoustic waves are generated in the tissue by laser energy, creating contrast in a manner unique from ultrasound, which is created by external acoustic energy focused on the tissue via ultrasound transducers.

Heterogeneous absorption of optical energy by tissues results in differential ultrasound signals that can yield both spatial and temporal information about the biological tissues being investigated. Structural information can be obtained, for example, from the fact that rapidly developing tumors consume more blood, and that the most malignant tumors have higher optical absorption (DiMarzio and Murray (2003) Subsurface Sensing Technologies and Applications 4(4):289-309). An example of how functional information can be conveyed is demonstrated in the photoacoustic spectra of oxygenated and deoxygenated hemoglobin, which differ in the near infrared range. Investigators are able to determine the state of oxygenation and may soon be able to detect differences in oxygen consumption across tissues. High scanning rates, high frequency probes and advances in various ultrasound imaging methods (for example, harmonic imaging, power spectrum, and three-dimensional imaging) are anticipated to allow faster imaging for screening and image analysis (Klibanov (2002) Topics in Current Chemistry 222:73-106.)

Photoacoustic imaging can also play a significant role in early detection and monitoring of cancer, for example in breast cancer. The normal breast is acoustically fairly homogenous, facilitating the photoacoustic detection of calcified or highly vascularized breast lesions. These structures may become more efficient absorbers of laser energy due to either increased blood flow or targeting via contrast agents. Photoacoustic breast imaging of suspicious lesions may prove a useful alternative or adjunct to X-ray mammography. An inherent advantage to photoacoustic detection of tumor masses is the generation of a photoacoustic signal without the need for using ionizing radiation or radioactive nuclides for detection.

The commercial markets of ultrasonic, optical and positron emission tomography (PET) imaging are potential markets for photoacoustic imaging. Prior to this time, optimization of imaging parameters such as sensitivity, spatial resolution, imaging depth, and contrast-to-noise ratio in a single imaging modality has been either unattainable or prohibitively costly. The general use of photoacoustic imaging methods has been limited to relatively thin biological samples because of the depth limitation of irradiation and signal attenuation.

The development of thermoacoustic and photoacoustic contrast agents, in conjunction with contrast agent-specific imaging equipment modifications, that can provide improved penetration compared to optical imaging techniques is of great clinical interest.

### SUMMARY OF THE INVENTION

Compositions of photoacoustic tomography (PAT) contrast agents, and methods of achieving contrast enhancement and amplification of photo-induced acoustic signal for in vivo photoacoustic imaging of animals and human subjects are provided. Such compositions and methods provide sensitivity comparable with optical and PET imaging without the use of radioactive contrast media, and significantly improved spatial resolution relative to ultrasound and PET imaging. Applications of the compositions and methods include the photoacoustic imaging of small animals for preclinical research and human lung or breast imaging systems for evaluating normal vs. disease states.

Contrast agents of interest are organic-based agents, which may be targeted or non-targeted, e.g. protein-based and/or lipid-based molecules, in combination with an inorganic core, e.g. a metal or silicon core, herein referred to as a composite PAT contrast agent. Preferred agents absorb in the near IR spectrum, e.g. from around about 650 nm to around about 800 nm, for example from about 740 to about 770 nm. In some embodiments of the invention, the organic component is a lipid composition, which optionally comprises one or more targeting moieties. Targeting moieties of interest include, without limitation, moieties that target blood vessel walls, which provide for unexpected enhancement of signal. In some embodiments the inorganic core is gold or another noble metal, e.g. silver, platinum, etc. The core may be nanoshells, nanospheres and nanorods, quantum dots, etc. The use of rods may provide for a desirable absorption spectrum.

The invention relates to a method of generating an image of an animate human or non-human animal body or part thereof. The method comprises administering to said body a contrast agent of the invention, exposing said body to radiation, e.g. laser light, microwaves, etc. in the range of from about 650 nm to about 800 nm, detecting pressure waves generated in said body by said radiation and generating a photocoustic image therefrom of at least a part of said body containing the administered contrast agent. Detectors may be externally applied, or applied at the end of an endoscope that will be put inside the body. The contrast agents may be used in combination with CMUT detectors for image signal detection and reconstruction.

The methods of the invention may further comprise administering a contrast agent that provide for a therapeutic benefit, e.g. by the delivery of a drug or polynucleotide. In other embodiments of the invention, the contrast agent is targeted to the tissue of interest for imaging. In some embodiments of the invention, the contrast agent is a composite nanoparticles of the invention. In some embodiments of the invention, the contrast agent is a cross-linked lipid nanoparticles with a noble metal core.

### BRIEF DESCRIPTION OF THE DRAWINGS

FIGS. 1A-1B are absorbance spectra of phosphocholine (A) and PDA (B) coated gold microspheres.

FIGS. 2A-2B are absorbance spectra of PDA (B) coated gold microrods.

FIG. 3 illustrates photoacoustic tomography using a cMUT transducer.

FIG. 4 illustrates the imaging of tube walls using gold, and PDA-coated gold contrast agents.

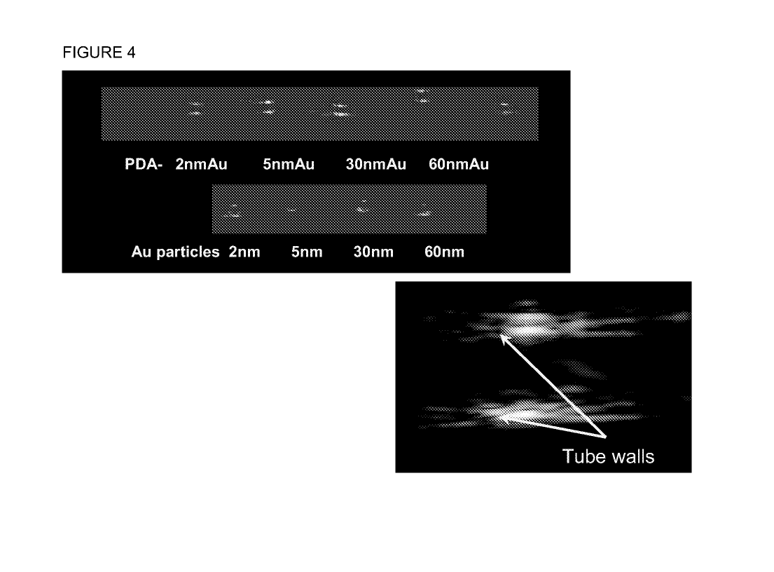

### DETAILED DESCRIPTION OF THE EMBODIMENTS

The invention provides contrast agents that are organic surface, e.g. a protein or lipid surface, in combination with an inorganic core, e.g. a metal or silicon core, herein referred to as a composite PAT contrast agent. Preferred agents absorb in the near IR spectrum, e.g. from around about 650 nm to around about 800 nm, for example from about 720 to about 790 nm. In some embodiments of the invention, the organic component is a lipid composition, which optionally comprises one or more targeting moieties. In some embodiments the inorganic core is gold or other noble metal, e.g. silver, platinum, etc. The core may be nanoshells, nanospheres and nanorods, quantum dots, etc. The use of rods may provide for a desirable absorption spectrum.

In some embodiments, the contrast agents have a gold core, e.g. a gold nanorod core, where the aspect ratio of the rod is usually at least 2, usually 3, and not more than about 6, usually not more than 5. Desirable aspect ratios include ratios of 3, 4 and 5. The diameter of the gold nanorod may range from about 2.5 nm to about 50 nm, usually from about 2.5 nm to about 25 nm. The surface of the contrast agent is an organic material, including lipids, as described herein, which may be modified to include a targeting moiety. Targeting moieties of interest include markers for blood vessels, e.g. proteins present on endotheiial cells.

Before the present compositions and methods are described are described in further detail, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

In biomedical photoacoustic imaging nanosecond laser pulses focused on a particular volume of tissue cause rapid heating and subsequent cooling which leads to thermoelastic tissue expansion and mechanical stress. When laser energy is focused upon the tissue H 0=E over A, light is scattered and absorbed μ_{eff}=√μₐ(μₐ+μ′ₐ). Photoacoustic stress generation is the result of the locally absorbed or deposited volumetric energy density W (x, y, z)=μₐ(x, y, z)H(x, y, z) producing a temperature increase ΔT (x, y, z)=W(x, y, z) over ρCₚ. Because the laser pulse duration is smaller than the propagation time of the pressure generated acoustic transient, instead of an initial volume expansion, a local stress transient is induced—pₒ(x, y, z)=Mβ over ρCₚ

(x, y, z)H(x, y, z). In satisfying conditions for stress confinement, efficient heat to pressure conversion and subsequent sound generation is possible.

H₀, surface radiant exposure [mJ/cm²]

E, laser pulse energy [mJ]

A, area [cm²]

ueff, effective attenuation coefficient [cm⁻¹]

μa, absorption coefficient [cm⁻¹]

μ's, reduced scattering coefficient [cm⁻¹]

W, energy density [J/m³]

ΔT, temperature increase [° C.]

ρ, density [kg/m3]

Cp, heat capacity [J/kg ° C.]

β, expansivity [strain per ° C.]

p₀, initial pressure [Pa]

M, bulk modulus [Pa per strain]

Ultrasound or cMUT transducers are used to detect the mechanically generated acoustic wave signals at the sample surface. The pressure field generated by the laser pulses and subsequently detected after interacting with heterogeneously absorbing and scattering tissue provides information about the spatial distribution of the absorbed electromagnetic energy. This permits mapping of the absorbed energy distribution within the tissue by its acoustic profile. The generation of sound waves by incident radiation is known as the “photoacoustic” or “optoacoustic” effect and is reviewed by Tam (Reviews of Modern Physics, 1986, 58(2), p381-431).

The incident radiation may be any type of energetic radiation, including electromagnetic radiation from radiofrequency to X-ray, electrons, protons, ions, and other particles. For simplicity, all of the above will be referred to herein as “radiation”. The word “light” will be used specifically to denote electromagnetic radiation of any wavelength or frequency. Preferred radiation is in the near IR spectrum, and may be generated by laser, microwave, etc.

Photoacoustic depth profiling can be performed when the measured sound wave is analysed in terms of transit time from the site of light absorption back to the detector. Signals from deep within a sample take longer to reach the detector than those from regions near the surface. For pulsed irradiation the longer transit time translates into a larger separation between the time of arrival of the pulse and the arrival of the signal at the detector. For amplitude-modulated irradiation, the longer transit time translates into a phase change in the detected sound wave. Together photoacoustic microscopy and photoacoustic depth profiling constitute photoacoustic imaging.

The use of short bursts of light rather than continuously applied light may be helpful for photoacoustic depth profiling. In this case, the absorption of each light pulse and subsequent heating of the various regions of the sample produces one or more positive or negative pressure waves that propagate radially from the site of absorption after each pulse. For very short light pulses, the shape of the pressure pulses generated by the light pulses is determined by the optical and thermal properties, sizes and shapes of the different regions of the sample, as well as by the speed of sound within the sites and the surrounding medium (see for example, Karabutov et al., 1996, Appl. Phys., 63, p545-563; Hutchins, 1986, Can. J. Phys., 64, p1247-1264).

Contrast agents permit light absorption and sound generation in regions not otherwise possible. Contrast agents may also improve signal:noise ratio by increasing the amplitude of the sound wave. Increasing the sound wave amplitude allows an increase in the possible maximum depth of detection and thereby allows imaging of objects further below the surface of the body.

The use of contrast media provides significant amplification of the signal strength, and thus permits improved imaging. Such a contrast agent for photoacoustic imaging works by either (i) enhancing the pre-existing photoacoustic effect or (ii) creating a photoacoustic effect where this was previously not possible. This may be achieved by selectively absorbing radiation in certain organs or healthy or diseased bodily structures or parts thereof, and/or by efficiently converting the radiation into heat, and/or by facilitating or improving heat-pressure conversion, and/or by scattering and diffusing the incident light so that it more uniformly illuminates the target organs.

Tissue of particular interest for imaging include, without limitation, tissues not shielded by bone, e.g. breast tissue, liver tissue: etc.; and blood vessels, which have been found to provide for unexpected amplification of signal. Subjects of interest for imaging include those suspected or know to have liver cancer, breast cancer, atherosclerosis, soft tissue sarcomas, and the like.

Composite Contrast Agents. Contrast agents of interest include agents having an organic surface material and an inorganic, usually metallic core. Agents typically have a diameter of from about 2.5 nm to about 250 nm. Where the core is a rod shape, the aspect ration is usually at least 2 and not more than 6, more usually at least 3 and not more than 5.

Core materials include metals, e.g. noble metals such as gold, silver, platinum, etc., and may also include composites thereof, e.g. gold plated silicon spheres, etc. The diameter of the core is usually from about 2 to about 100 nm, more usually from about 2 to about 50 nm, and may be from about 2 to about 25 nm. Various geometries may be used, e.g. hollow or solid cores shapes as spheres, tubes, rods, etc. Gold rods are of particular interest. Such rods may be synthesized by various methods known in the art, e.g. as reviewed by Perez-Juste et al. (2005) Coordination Chemistry 249:1870-1901, herein specifically incorporated by reference.

The inorganic core is combined with an organic coating, e.g. an amphipathic coating, such as lipids. The agents are self-assembled aggregates of amphipathic molecules with the inorganic core. Optionally the aggregate cross-linked. The amphipathic molecules may include cationic molecules, neutral molecules, and targeting molecules, where a targeting molecule comprises a targeting moiety, usually a targeting moiety attached to a head group.

Suitable amphipathic molecules have a structure as shown below, comprising a hydrophilic head group, which may be a chemically reactive head group; a linker or covalent bond between the head and tail groups; and a hydrophobic tail group for self-assembly into nanoparticles. The molecules may comprise a cross-linking group, which cross-linking group may comprise all or part of the tail group and/or the linker. A mixture of molecules may provide different functional groups on the hydrophilic exposed surface. For example, some hydrophilic head groups may have functional surface groups, for example, biotin, amines, cyano, carboxylic acids, isothiocyanates, thiols, disulfides, α-halocarbonyl compounds, α,β-unsaturated carbonyl compounds and alkyl hydrazines for attachment of targeting moieties.

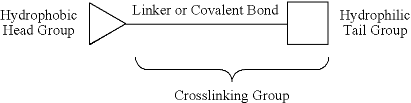

Amphiphilic molecules suitable for constructing targeting nanoparticles have a hydrophilic head group and a hydrophobic tail group, where the hydrophobic group and hydrophilic group are joined by a covalent bond, or by a variable length linker group. The linker portion may be a bifunctional aliphatic compounds which can include heteroatoms or bifunctional aromatic compounds. Preferred linker portions include, e.g. variable length polyethylene glycol, polypropylene glycol, polyglycine, bifunctional aliphatic compounds, for example amino caproic acid, or bifunctional aromatic compounds.

Amphipathic molecules of interest include lipids, which group includes fatty acids, neutral fats such as triacylglycerols, fatty acid esters and soaps, long chain (fatty) alcohols and waxes, sphingoids and other long chain bases, glycolipids, sphingolipids, carotenes, polyprenols, sterols, and the like, as well as terpenes and isoprenoids. For example, molecules such as diacetylene phospholipids may find use as neutral amphipathic molecules.

In some embodiments at least a portion, e.g. from about 50% to about 95% or more, up to 99% or more of the amphipathic molecules are phosphocholine. In other embodiments at least a portion e.g. from about 50% to about 95% or more, up to 99% or more of the amphipathic molecules are a diacetylene phospholipid, e.g. 10,12-pentacosadiynoic acid or a derivative thereof, e.g. N-(11-O-R-D-Mannopyranosyl-3,6,9-trioxa)undecyl 10,12-pentacosa-diynamide (PDTM), etc., see Kim et al. (2005) Macromolecular Research. Vol. 13, No. 3, pp 253-256, U.S. Pat. No. 6,866,863, and the like, as known in the art.

The size of the nanoparticles can be controlled, e.g. by extrusion, sonication, etc. Preferably the nanoparticles are at least about 2.5 nm in diameter and not more than about 250 nm in diameter, more usually at least about 35 nm in diameter and not more than about 100 nm in diameter, and may be from about 40 nm in diameter to from about 50 nm in diameter.

The component amphipathic molecules of the contrast agents of this invention may be purified and characterized individually using standard, known techniques and then combined in controlled fashion to produce the final particle.

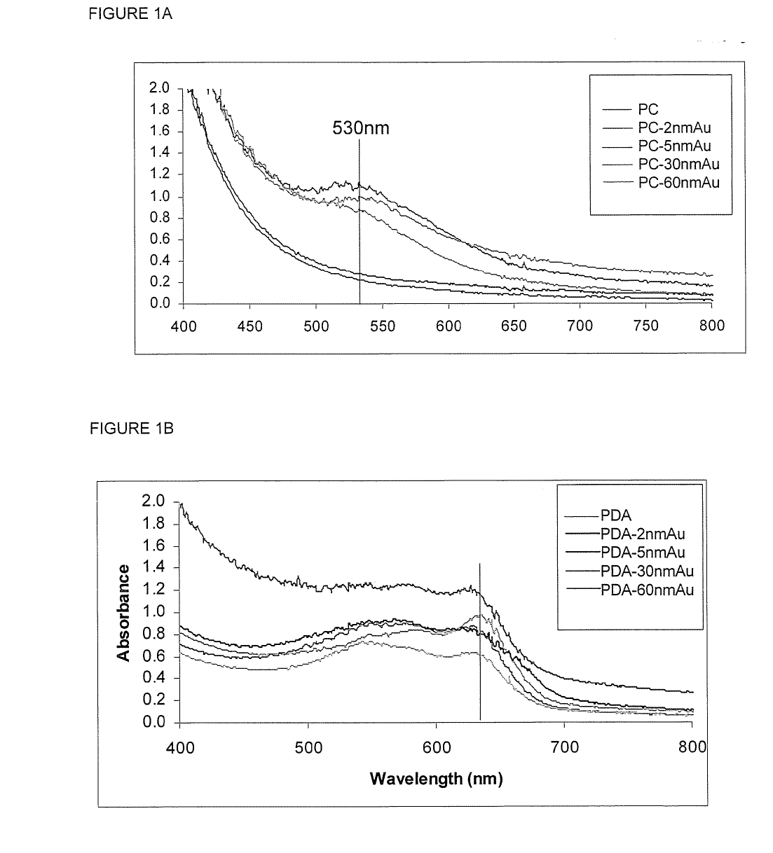

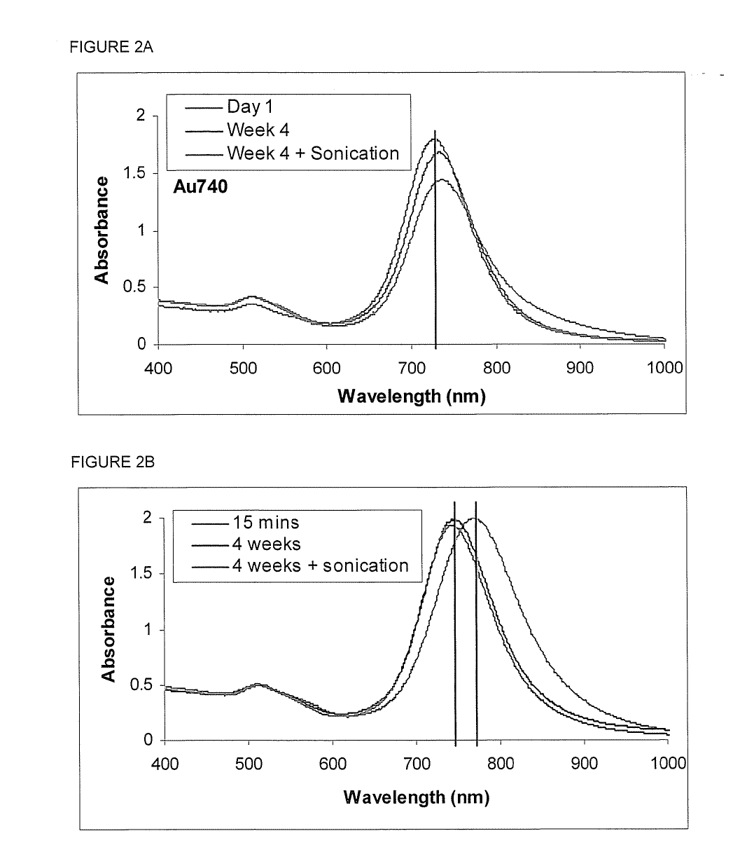

Preferred contrast agents have a peak absorption of at least 600 nm, usually at least about 700 nm, and preferably at least about 740 nm. As shown in FIG. 1A, contrast agents having a gold microsphere as a core, and phosphocholine surface, have an absorption peak at about 530 nm. The absorption is shifted up by the use of PDA (10,12-pentacosadiynoic acid) on the surface, shown in FIG. 1B. When a gold nanorod having an aspect ration between 3 and 5 is used as the core, as shown in FIGS. 2A and 2B, with a surface of PDA, the absorbance peak is desirably shifted to 740-770 nm.

A targeting amphipathic molecule has the structure as described above, comprising a hydrophilic and a hydrophobic group, and further comprises a targeting moiety, usually a targeting moiety covalently or non-covalently bound to the hydrophilic head group. Head groups useful to bind to targeting moieties include, for example, biotin, amines, cyano, carboxylic acids, isothiocyanates, thiols, disulfides, α-halocarbonyl compounds, α,β-unsaturated carbonyl compounds, alkyl hydrazines, etc. The amphipathic molecule provides a component of the cross-linked nanoparticle, and the bound targeting moiety resides on the exterior of the nanoparticle, where it is accessible for interaction. Preferably the targeting moiety is bound to an amphipathic molecule prior to synthesis of the contrast agent, however in some cases the targeting moiety will be added to preformed contrast agents.

Chemical groups that find use in linking a targeting moiety to an amphipathic molecule also include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

The linkage between targeting moiety and amphipathic molecules may comprise spacers, e.g. alkyl spacers, which may be linear or branched, usually linear, and may include one or more unsaturated bonds; usually having from one to about 300 carbon atoms: more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O)ₙ where n is from 1 to about 12; (CH₂CH₂NH)ₙ, where n is from 1 to about 12; [(CH₂)ₙ(C═O)NH(CH₂)m]z, where n and m are from 1 to about 6, and z is from 1 to about 10; [(CH₂)ₙOPO₃(CH₂)ₘ]_{z} where n and m are from 1 to about 6, and z is from 1 to about 10. Such linkers may include polyethylene glycol, which may be linear or branched.

The targeting moiety may be joined to the amphipathic molecule through a homo- or heterobifunctional linker having a group at one end capable of forming a stable linkage to the hydrophilic head group, and a group at the opposite end capable of forming a stable linkage to the targeting moiety. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate. N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N,N′-(1,3-phenylene) bismaleimide; N,N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m.m′-dinitrodiphenylsulfone (which forms irreversible cross-linkages with amino and phenolic groups): dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups): disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

For example, targeting molecules may be formed by converting a commercially available lipid, such as DAGPE, a PEG-PDA amine, DOTAP, etc. into an isocyanate, followed by treatment with triethylene glycol diamine spacer to produce the amine terminated thiocarbamate lipid which by treatment with the para-isothiocyanophenyl glycoside of the targeting moiety produces the desired targeting glycolipids. This synthesis provides a water soluble flexible linker molecule spaced between the amphipathic molecule that is integrated into the contrast agent, and the ligand that binds to cell surface receptors, allowing the ligand to be readily accessible to the protein receptors on the cell surfaces.

To obtain selectivity, the contrast agent may be passively or actively targeted to regions of diagnostic interest such as organs, vessels, sites of disease, tumorous tissue, or a specific organism in a patient. In active targeting, the contrast agents may be attached to biological recognition agents to allow them to accumulate in or to be selectively retained by or to be slowly eliminated from certain parts of the body, such as specific organs, parts of organs, bodily structures and disease structures and lesions. Active targeting is defined as a modification of biodistribution using chemical groups that will associate with species present in the desired tissue or organism to effectively decrease the rate of loss of contrast agent from the specific tissue or organism.

Active targeting of a contrast agent can be considered as localization through modification of biodistribution of the contrast agent by means of a targeting chemical group or ligand that is attached to or incorporated into the contrast agent. The ligand or targeting group can associate or bind with one or more receptor species present in the tissue or organism of diagnostic interest. This binding will effectively decrease the rate of loss of contrast agent from the specific tissue or organism of diagnostic interest. In such cases, the contrast agent can be modified synthetically to incorporate the targeting ligand or targeting vector. Targeted contrast agents can localize because of binding between the ligand and the targeted receptor. Alternatively, contrast agents can distribute by passive biodistribution, i.e., by passive targeting, into diseased tissues of interest such as tumors. Thus, even without synthetic manipulation to incorporate a targeting ligand or vector that can bind to a receptor site, passively targeted contrast agents can accumulate in a diseased tissue or in specific locations in the patient such as the liver. The present invention comprises use of a contrast agent that is linked to a targeting vector (also referred to as a ligand) that has an affinity for binding to a receptor. Preferably the receptor is located on the surface of a diseased or disease-causing cell in a human or animal patient.

A targeting moiety, as used herein, refers to all molecules capable of specifically binding to a particular target molecule and forming a bound complex as described above. Thus the ligand and its corresponding target molecule form a specific binding pair.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

Examples of targeting moieties include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting moieties of particular interest include peptidomimetics, peptides, antibodies and antibody fragments (e.g. the Fab′ fragment). For example, β-D-lactose has been attached on the surface to target the aloglysoprotein (ASG) found in liver cells which are in contact with the circulating blood pool.

Cellular targets include tissue specific cell surface molecules, for targeting to specific sites of interest, e.g. neural cells, liver cells, bone marrow cells, kidney cells, pancreatic cells, muscle cells, and the like. For example, nanoparticles targeted to hematopoietic stem cells may comprise targeting moieties specific for CD34, ligands for c-kit, etc. Nanoparticles targeted to lymphocytic cells may comprise targeting moieties specific for a variety of well known and characterized markers, e.g. B220, Thy-1, and the like.

Endothelial cells are a target of particular interest, in particular endothelial cells found in blood vessels, e.g. during angiogenesis, inflammatory processes, and the like. Among the markers present on endothelial cells are integrins, of which a number of different subtypes have been characterized. Integrins can be specific for endothelial cells involved in particular physiological processes, for example certain integrins are associated with inflammation and leukocyte trafficking (see Alon & Feigelson (2002) Semin Immunol. 14(2):93-104; and Johnston & Butcher (2002) Semin Immunol 14(2):83-92, herein incorporated by reference). Targeting moieties specific for molecules such as ICAM-1, VCAM-1, etc. may be used to target vessels in inflamed tissues.

Endothelial cells involved in angiogenesis may be targeted for site directed delivery of nucleic acids. Diseases with a strong angiogenesis component include tumors growth, particularly solid tumor growth, psoriasis, macular degeneration, rheumatoid arthritis, osteoporosis, and the like. A marker of particular interest for angiogenic endothelial cells is the αvβ3 integrin. Ligands for this integrin are described, for example, in U.S. Pat. Nos. 5,561,148: 5,776,973; and 6,204,280; and in International patent publications WO 00/63178; WO 01/10841; WO 01/14337; and WO 97/45137, herein incorporated by reference.

The amphipathic molecules optionally comprise a crosslinking functional group, e.g. diacetylene, olefins, acetylenes, nitrites, alkyl styrenes, esters, thiols, amides, αβunsaturated carbonyl compounds, etc. in the linker or tail group of the molecule. The cross-linking groups irreversibly cross-link, or polymerize, when exposed to ultaviolet light or other radical, anionic or cationic, initiating species, while maintaining the distribution of functional groups at the surface of the contrast agent. The cross-linking functional groups may be located at specific positions on hydrophobic portion of the amphipathic molecule.

After initiation of cross-linking, oligomers of at least two and not more than about 100 monomeric amphipathic molecules are formed, usually at least two and not more than about 30 monomers are present in the cross-linked oligomer.

Cationic amphipathic groups include any amphiphilic molecule as described above, including lipids, synthetic lipids and lipid analogs, having hydrophobic and hydrophilic moieties, a net positive charge, and which by itself can form spontaneously into bilayer vesicles or micelles in water. The term also includes any amphipathic molecules that can be stably incorporated into lipid micelle or bilayers in combination with phospholipids, with its hydrophobic moiety in contact with the interior, hydrophobic region of the micelle or bilayer membrane, and its polar head group moiety oriented toward the exterior, polar surface of the membrane.

The term “cationic amphipathic molecules” is intended to encompass molecules that are positively charged at physiological pH, and more particularly, constitutively positively charged molecules, comprising, for example, a quaternary ammonium salt moiety. Cationic amphipathic molecules used for gene delivery typically consist of a hydrophilic polar head group and lipophilic aliphatic chains. Similarly, cholesterol derivatives having a cationic polar head group may also be useful. See, for example, Farhood et al. (1992) Biochim. Biophys. Acta 1111:239-246: Vigneron et al. (1996) Proc. Natl. Acad. Sci. (USA) 93:9682-9686.

Cationic amphipathic molecules of interest include, for example, imidazolinium derivatives (WO 95/14380), guanidine derivatives (WO 95/14381), phosphatidyl choline derivatives (WO 95/35301), and piperazine derivatives (WO 95/14651). Examples of cationic lipids that may be used in the present invention include DOTIM (also called BODAI) (Solodin et al., (1995) Biochem. 34: 13537-13544), DDAB (Rose et al., (1991) BioTechniques 10(4):520-525), DOTMA (U.S. Pat. No. 5,550,289), DOTAP (Eibl and Wooley (1979) Biophys. Chem. 10:261-271), DMRIE (Felgner et al., (1994) J. Biol. Chem. 269(4): 2550-2561), EDMPC (commercially available from Avanti Polar Lipids, Alabaster, Ala.), DCChol (Gau and Huang (1991) Biochem. Biophys. Res. Comm. 179:280-285), DOGS (Behr et al., (1989) Proc. Natl. Acad. Sci. USA, 86:6982-6986), MBOP (also called MeBOP) (WO 95/14651), and those described in WO 97/00241. In addition, contrast agents having more than one cationic species may be used to produce complexes according to the method of the present invention.

Synthesis of contrast agents. To synthesize targeting nanoparticies, the component amphipathic molecules and inorganic core are mixed in an aqueous environment. Typically the amphipathic molecules are dehydrated, and rehydrated in the presence of the inorganic core. The components are mixed, usually with the application of energy, e.g. laser light, and are allowed to self-assemble.

The contrast agents are formulated in a pharmaceutically acceptable excipient, such as wetting agents, buffers, disintegrants, binders, fillers, flavoring agents and liquid carrier media such as sterile water, water/ethanol etc. The contrast agent should be suitable for administration either by injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range, e.g. 2 to 7, and buffers or pH adjusting agents may be used. The contrast media may be formulated in conventional pharmaceutical administration forms, such as tablets, capsules, powders, solutions, dispersion, syrups, suppositories etc.

The preferred dosage of the contrast media will vary according to a number of factors, such as the administration route, the age, weight and species of the subject, but in general containing in the order of from 1 μmol/kg to 1 mmol/kg bodyweight of the contrast agent.

Administration may be parenteral (e.g. intravenously, intraarterially, intramuscularly, interstitially, subcutaneously, transdermally, or intrasternally) or into an externally voiding body cavity (e.g. the gastrointestinal tract, bladder, uterus, vagina, nose, ears or lungs), in an animate human or non-human (e.g. mammalian, reptilian or avian) body. Usually administration is accomplished by intravenous or intratumor injection.

Imaging of the desired area is performed by detection and appropriate analysis of the sound waves resulting from irradiation. Detection may be performed at the same surface of the sample as the source of incident radiation (reflection) or alternatively at another surface such as the surface diametrically opposed to the incident light, i.e. the sample's back surface (transmission). Suitable methods of detection include the use of a microphone, piezoelectric transducer, capacitance transducer, fiber-optic sensor, cMUT, or alternatively non-contact methods (see Tam, 1986, supra for a review). Techniques and equipment used in ultrasound imaging may be used.

The methods and uses described herein are especially useful for imaging blood-containing structures, e.g. blood vessels, which may be present in tumours, diseased tissue or particular organs, by the use of contrast agents with specificity for that region/structure, e.g. by use of biological recognition agents with the desired specificity.

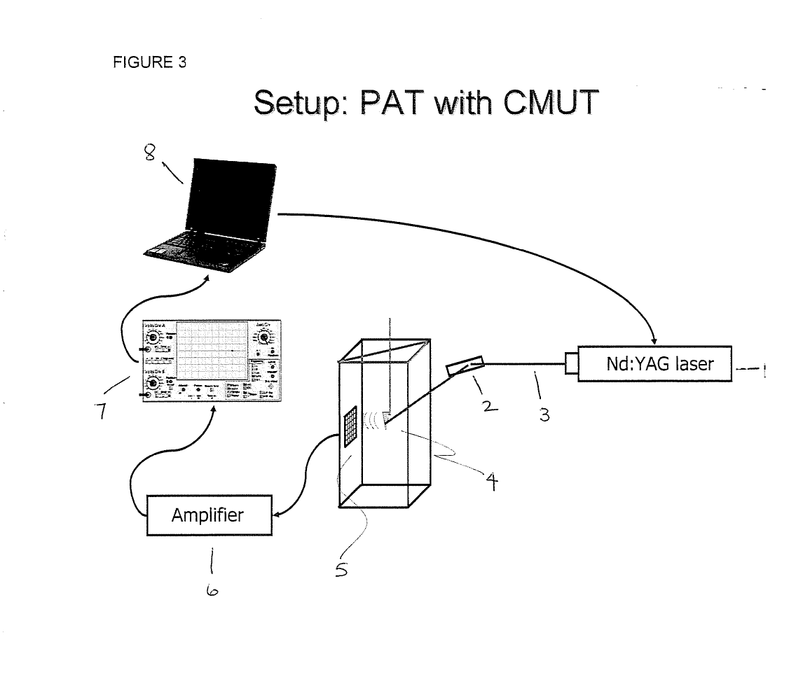

As shown in FIG. 3, imaging may be performed with a cMUT transducer (see, for example, see Ozevin et al. (2005) Ultrasonics Symposium, 2005 IEEE Volume: 2, pp: 956-959, herein specifically incorporated by reference. A laser 1 provides a beam 3 that can be deflected 2 to irradiate a contrast agent 4, which may be present in a test sample or apparatus, or in a living organism, 4. The ultrasound waves thus produced are detected with a transducer 5, for example a cMUT transducer. The signals thus received are amplified 6, 7 and transmitted to a data processor for analysys 8, which data processor optionally controls the laser 1.

Continuous wave radiation may be used with its amplitude or frequency modulated. When continuous wave radiation is used, the photoacoustic effects may be analysed in the frequency domain by measuring amplitude and phase of one or several Fourier components. Alternatively, and preferably, short pulses (impulses) of radiation are employed which allow stress confinement. When pulses are used, analysis may be made in the time domain, i.e. on the basis of the time taken for the sound wave to reach the detector, thus simplifying analysis and aiding depth profiling.

The following examples are put forth so as to provide those of ordinary skill in the art with a complete disclosure and description of how to carry out the invention and are not intended to limit the scope of what the inventors regard as their invention, nor are they intended to represent or imply that the experiments below are all of or the only experiments performed. Efforts have been made to ensure accuracy with respect to numbers used (e.g., amounts, temperatures, etc.), but some experimental error and deviation should be accounted for. Unless indicated otherwise, parts are parts by weight, molecular weight is weight average molecular weight, and temperature is in degrees Centigrade.

### EXPERIMENTAL

### Example 1

### Synthesis of Composite Contrast Agents

### Methods

Lipids used in synthesis of contrast agents are:

phosphocholine

PDA

and optionally include 1-10% biotinylated lipid.

The lipid solutions are evaporated to dryness and dried under high vacuum to remove any residual solvent. The dried lipid film is hydrated to a known lipid density (15-30 mM) using deionized water, in combination with the inorganic core particles. The resulting suspension is then sonicated at temperatures above the gel-liquid crystal phase transition (Tm @ 64° C.) for 1 hr. using a probe-tip sonicator while maintaining the pH between 7.0 and 7.5 using a 0.1 M sodium hydroxide solution. The solution can be sterile filtered through 0.2 mm filter and stored under argon at room temperature.

An avidin/antibody complex, using an LM609 antibody, which is specific for the integrin αᵥβ₃ (see Sipkins et al., (1998) Nat. Med. 4:623-626) is combined with particles having biotinylated lipid in a ratio of 1.4 mg antibody to 1 ml particle and incubated overnight at 4° C. Alternatively, a non-peptidic integrin antagonist as described by Cheresh et al. (2002) Science 296:2404-2407

What is claimed is:

1. A contrast agent for photoacoustic tomography, comprising:
a particle of from 2.5 to 250 nm diameter having an inorganic core and an organic surface, wherein the particle has an absorption peak from 650 to 800 nm wavelength;
and a pharmaceutically acceptable excipient.

2. The contrast agent of claim 1, wherein the inorganic core is a metallic core of from 2.5 to 50 nm diameter.

3. The contrast agent of claim 1, wherein the inorganic core is a noble metal.

4. The contrast agent of claim 3, wherein the inorganic core is gold.

5. The contrast agent of claim 4, wherein the gold core is a nanorod of from 2.5 to 50 nm diameter, having an aspect ratio between 2 and 6.

6. The contrast agent of claim 5, wherein the nanorod has an aspect ratio between 3 and 5.

7. The contrast agent of claim 6, wherein the particle has an absorption peak from 720 to 800 nm wavelength.

8. The contrast agent of claim 7, wherein the particle has an absorption peak from 740 to 770 nm wavelength.

9. The contrast agent of claim 3, wherein the core is complexed with a lipid.

10. The contrast agent of claim 9, wherein the lipid comprises at least 50% diacetylene phospholipid.

11. The contrast agent of claim 10, wherein the core is a gold nanorod.

12. The contrast agent of claim 10, wherein the lipid further comprises at least one targeting lipid.

13. The contrast agent of claim 12, wherein the targeting lipid comprises a targeting moiety specific for a blood vessel marker.

14. An imaging method, comprising contacting a biological material with a composition set forth in claim 1;
exposing the biological material to irradiation at a wavelength between 650 and 800 nm:
transducing the resulting ultrasound signal from the biological material:
producing an image in a data processor from the transduced ultrasound signal.

15. The method of claim 14, wherein the biological material is a blood vessel.

16. The method of claim 15, wherein he blood vessel is present in a mammal.
